(19) United States
(12) Patent Application Publication
(10) Pub. No.: US 2015/0134347 A1
(43) Pub. Date: May 14, 2015

(54) SELF-SERVE KIOSK FOR BLOOD /FLUID/TISSUE COLLECTION AND SCREENING

(71) Applicant: LabATM INC., Solana Beach, CA (US)

(72) Inventors: Philippe FAURIE, San Diego, CA (US); Pieter Van Rooyen, San Diego, CA (US)

(73) Assignee: LabATM INC., Solana Beach, CA (US)

(21) Appl. No.: 14/283,485

(22) Filed: May 21, 2014

Related U.S. Application Data
(60) Provisional application No. 61/827,441, filed on May 24, 2013.

(51) Int. Cl. G06F 19/00; A61B 5/15; A61B 5/00; A61B 5/021; A61B 5/024; A61B 5/1455; A61B 10/02; A61B 5/0205

(52) U.S. Cl. CPC G06F 19/3431; A61B 10/02; A61B 5/150755; A61B 5/0205; A61B 5/021; A61B 5/024; A61B 5/14552; A61B 5/72; G06Q 40/08
USPC 705/2; 600/562; 600/573; 600/301

(57) ABSTRACT

A self-serve kiosk includes a blood, fluid or tissue test device for obtaining a blood, fluid or tissue sample from a user, analyzing the sample, and generating in vitro diagnostic data. The kiosk may also have non-invasive test devices for obtaining vitals measurements from the user, such as a weight scale, a blood pressure device and a heart rate monitor. A user interface such as a touch screen obtains input of personal health information from the user. The test measurements and input data are analyzed, and a health snapshot, risk assessment, guidance plans and incentives are generated and provided to the user.

### CROSS-REFERENCE TO RELATED APPLICATIONS

This application claims the priority of U.S. provisional application Ser. No. 61/827,441, filed on May 24, 2013.

### BACKGROUND OF THE INVENTION

This invention is in the field of preventative health care, and provides individuals with a unique platform to measure, self-monitor and/or improve their health condition.

### SUMMARY OF THE INVENTION

The platform of the present invention provides a kiosk as a unique collecting point for a user to import personal information, such as personal health information, vital signs, lifestyle information and nutritional information, fluid samples, such as blood, saliva and urine, and tissue samples, such as hair. The personal information, blood/fluid and/or tissue samples imported by the kiosk user are combined and analyzed, and feedback is provided to the individual in the form of test results, risk assessments, guidance and incentives for better health. In addition, the kiosk may generate and output recommended services, products, coupons and/or cash, based on the data input by the kiosk user and the test results, that may improve the health of the kiosk user.

FIG. 1 is a conceptual diagram generally illustrating the principles of the present invention. The invention provides a personalized platform 110 for personal prevention that receives various inputs 120 from a user and generates outputs 130 that may be useful to the user as well as other entities. As will be described below with reference to FIG. 3, platform 110 may include kiosk 1, server 2 and user device (i.e. smart phone, tablet or computer) 3. Inputs 120 provided by the kiosk user include, without limitation, vitals 122 such as blood pressure (BP), weight, heart rate, height, oxygen levels and other personal health information; IVD (in vitro diagnostic) data from blood/fluid/tissue samples collected from the user such as cholesterol and glucose levels; genomic/genetic/molecular data 126 obtained from more complex analysis of the user's blood/fluid/tissue samples; and personal health data 128 such as age, nutritional profiles, exercise habits, lifestyle data, electronic health records, etc.

Based on input data 120, platform 110 generates output data 130 of use to the kiosk user as well as other entities. Feedback 132 is provided to the individual in the form of health snapshots (test results), risk assessments, guidance and incentives for better health. The output data generated by platform 110 may also be useful to research and development in the medical, pharmaceutical and other industries 134. Payers 136 may find output data 130 useful for purposes such as health care cost optimization. In addition, with respect to the retail sector 138, the platform of the present invention may generate and output recommended services, products, coupons and/or cash, based on the user input data and the test results, that may improve the health of the kiosk user.

FIG. 2 is a conceptual diagram generally illustrating the platform of the present invention from the consumer perspective. A consumer (kiosk user) 150 provides vitals measurements and 152, personal health information (PHI) 154 and blood/fluid/tissue samples 156 and possible genomic data derived therefrom, to platform 160. In response, platform 160 provides to user 150 results and risks 162 that consumer 150 can then monitor (164). In addition, platform 160 provides guidance 166, such as exercise and health plans, that consumer 150 can utilize to change and improve his/her health (168).

Other features and advantages of the invention will be apparent from the following detailed description, taken in conjunction with the accompanying drawings that illustrate, by way of example, various features of the invention.

### BRIEF DESCRIPTION OF THE DRAWINGS

FIG. 1 is a conceptual diagram generally illustrating the principles of the present invention.

FIG. 2 is a conceptual diagram generally illustrating the platform of the present invention from the consumer perspective.

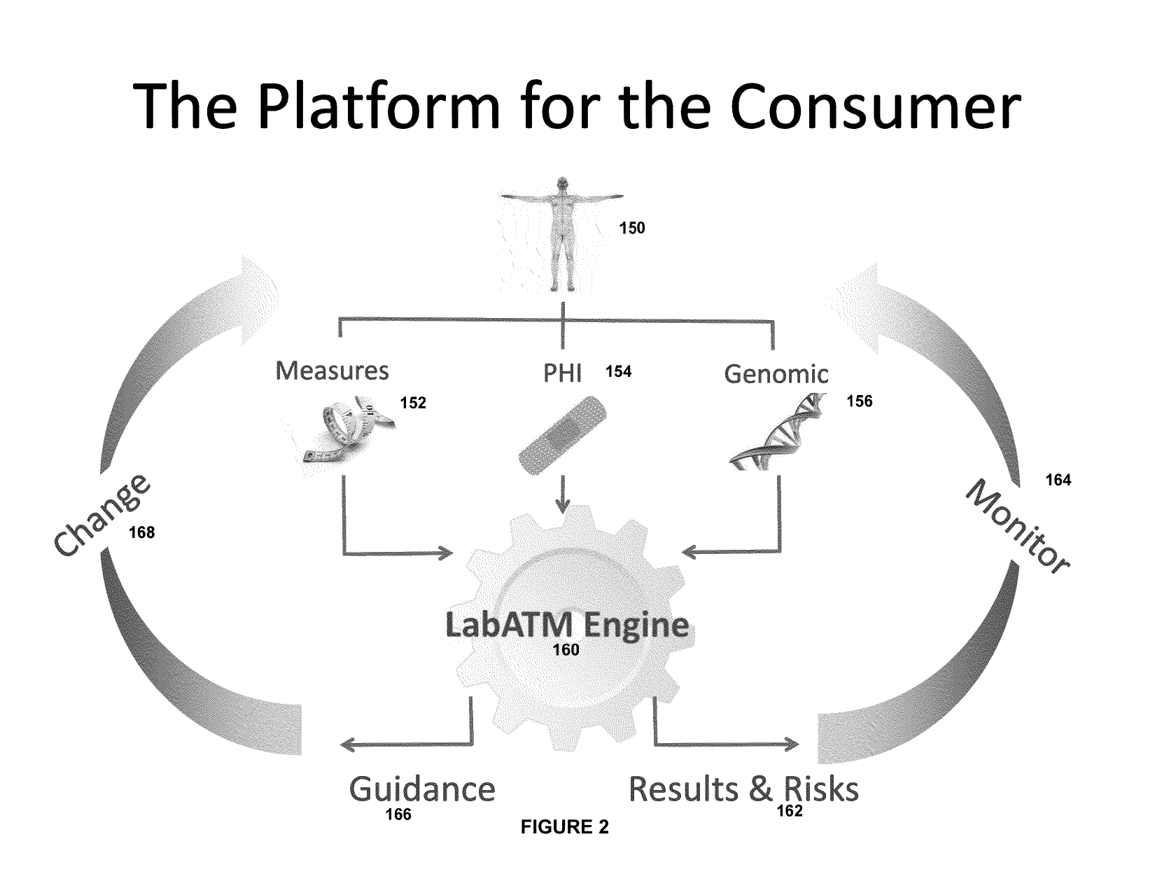

FIG. 3 is a block diagram of the system architecture of the platform of the present invention.

FIG. 4 is a block diagram of a kiosk of the present invention.

FIG. 5 is a block diagram showing additional components of the system architecture of the present invention.

FIG. 6 is a flow chart illustrating the algorithm implemented by the kiosk software according to the present invention.

FIG. 7 is a conceptual diagram showing the data collection and flow that occurs during execution of the algorithm of FIG. 6.

FIG. 8 is a block diagram of a general purpose computing environment in which the present invention may be implemented.

### DETAILED DESCRIPTION OF THE INVENTION

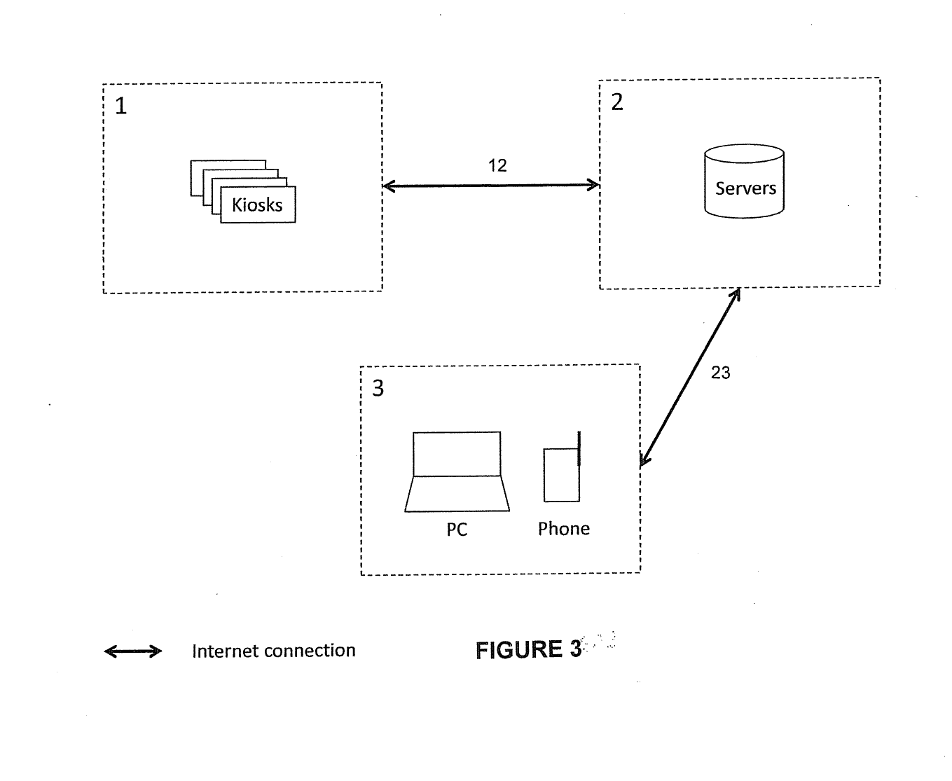

FIG. 3 is a block diagram showing the system architecture of the platform of the present invention. As shown in FIG. 3, the platform of the present invention is comprised of three sub-systems interconnected through the Internet (wired or wireless connection): kiosk(s) 1; servers and infrastructure 2; and user device 3 such as a personal computer (PC), tablet computer or smart phone. As shown in FIG. 3, kiosk 1 and server 2 are connected by Internet connection 12, and server 2 and user device 3 are connected by Internet connection 23.

Kiosk 1 is used by a user to import fluid and tissue samples, non-invasive test measurement data and personal health data. Kiosk 1 is preferably placed in high traffic venues such as malls, grocery stores, gyms and other locations that are convenient and easily accessible.

Kiosk 1 includes a blood/fluid/tissue collector that collects blood/fluid/tissue from the kiosk user into a container. An example of such a blood/fluid/tissue collector includes, without limitation, a finger stick device that pricks the finger of the kiosk user. Kiosk 1 retrieves the container for analysis and measurement of the collected blood/fluid/tissue. For example, in vitro diagnostic (IVD) data such as cholesterol and glucose levels may be measured and analyzed from a blood sample. Kiosk 1 may include additional devices for collection of other fluids such as saliva, urine, etc., and for collection of tissue samples such as hair, etc. The collected blood/fluid/tissue samples may be preserved for further analysis and measurement of data such as genetic data.

Kiosk 1 may also be equipped with a set of non-invasive test devices that can be used by the kiosk user to import vitals measurement data. Examples of such devices include, without limitation, weight scales, heart rate monitors/oxymeters and blood pressure devices.

Kiosk 1 receives direct import of data from the consumer such as personal health information, lifestyle information and nutritional profile information. The data may be input directly into kiosk 1 via a keyboard/mouse or touchscreen, or alternatively, may be input remotely from user device 3 via Internet connection 23 with server 2. Additional data may be obtained by kiosk 1 and/or server 2 and synchronized from external sources such as medical record systems and retailer member log systems.

The data obtained by kiosk 1 is combined and analyzed by software executed by a processor residing on kiosk 1 and/or server 2. Output may be generated directly at kiosk 1 and provided to the kiosk user. For example, based on the test results and data input by the user, kiosk may output a health snapshot (test results), risk assessment and lifestyle change recommendations such as exercise and diet plans. In addition, recommendations of products and services, and coupons of benefit to the kiosk user's health may be output by kiosk 1.

The data obtained by kiosk 1 is also provided to server 2 via Internet connection 12. The data may also be combined and analyzed by server 2, and the results along with possible guidance for lifestyle or diet change provided to user device 3 via Internet connection 23. For example, user device 3 may be a smart phone or tablet executing a mobile application (“app”) that displays the data received from server 2 and provides a number of options. Options may include, without limitation, display of current test results on user device 3; display of test result history on user device 3; sharing information with a medical network or a physician; storage of data in a personal health record; making an appointment with or calling a physician or health care expert, who will be able to access the recorded health record of the consumer; and display of advertisements related to the consumer's health profile.

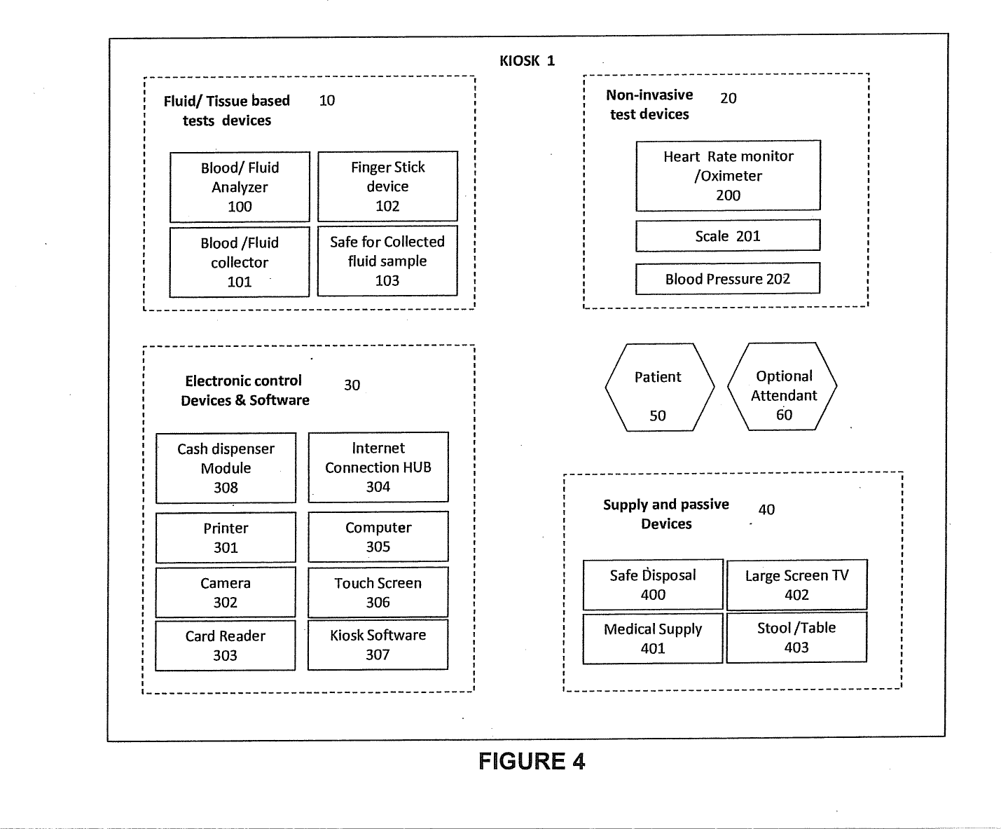

FIG. 4 is a block diagram of kiosk 1. As shown in FIG. 4, kiosk 1 comprises fluid/blood/tissue test devices 10; non-invasive test devices 20; electronic control devices and software 30; and supply and passive devices 40. In addition, an attendant 60 may optionally be present at kiosk 1 in order to assist patient 50 in operating kiosk 1, and to maintain and service the kiosk.

The fluid/blood/tissue test devices 10 of kiosk 1 may include, without limitation, a blood/fluid/tissue collection device 102 that automatically draws a small amount of blood, fluid or tissue from kiosk user (patient) 50 (a finger stick device such as a lancet is one example); a blood/fluid/tissue collector 101 for collecting the blood/fluid/tissue collected from kiosk user 50; a blood/fluid/tissue analyzer 100 into which collector 101 is automatically placed; and a safe 103 into which collected blood/fluid/tissue samples are placed for later collection and shipment to an external professional laboratory. Kiosk 1 may include one or more of each of collection device 102, collector 101, analyzer 100 and safe 103. When present, though not required, an attendant 60 such as a qualified phlebotomist or nurse may assist the kiosk user in using collection device 102 and placing the collected blood/fluid/tissue into collector 101 and analyzer 100. As will be described herein, kiosk 1 also includes a safe disposal 400 into which contaminated collection devices 102, collectors 101 and other used medical supplies are safely disposed.

The non-invasive test devices 20 of kiosk 1 may include, without limitation, heart rate monitor/oximeter 200 to measure heart rate and oxygen level; weight scale 201 to measure weight; and blood pressure device 202 to measure blood pressure. One or more of each of these devices may be present in kiosk 1, and kiosk 1 may also include other non-invasive test devices.

The electronic control devices and software 30 of kiosk 1 may include, without limitation, printer 301; camera 302; card reader 303; Internet connection hub 304; computer 305 including a processor; touch screen 306; and kiosk software 307. The kiosk user interacts with kiosk 1 via touch screen 306, with or without the help of optional attendant 60. The kiosk user identifies himself or herself via touch screen 306, and is guided through the process of performing selected tests by touch screen 306 and kiosk software 307. Though kiosk 1 may be used anonymously, some form of contact information such as an email address or mobile phone number must be provided in order for test results to be pushed electronically to the kiosk user. In addition, although a touch screen 306 is described, kiosk 1 may include alternative or additional user input mechanisms including, without limitation, keyboard, mouse or voice command input mechanisms.

If payment or insurance verification is required, the kiosk uses card reader 303 when prompted by touch screen 306 to scan insurance and/or payment information. Touch screen 306 and card reader 303 are connected to computer 305 that controls the transaction with online third party verification software accessed through Internet connection hub 304 or through a direct Internet connection. While the kiosk user interacts with touch screen 306, camera 302 captures the event and a video stream of the encounter is accessible by a remote kiosk operation center in real time. Touch screen 306, camera 302 and computer 305 may, in one implementation, be integrated into a single device such as a tablet.

Printer 301 is connected to computer 305 and allows the kiosk user to print hard copies of the test measurements and results. In addition, printer 301 may print information such as health risk assessments and lifestyle change recommendations such as exercise and diet plans. Kiosk 1 is also equipped with cash dispenser module 308. In order to entice use of kiosk 1, cash dispenser module 308 provides the ability to distribute rewards such as printed coupons/vouchers or cash. For example, coupons or vouchers that are pertinent to the lifestyle changes recommended by kiosk 1 may be distributed to the kiosk user.

The supply and passive devices 40 of kiosk 1 may include, without limitation, safe disposal 400, medical supplies 401, large screen TV 402 and stool/table 403. Medical supplies 401 may include, without limitation, fluid/blood/tissue collection devices, swabs, containers, lancets and bandages that are used to perform tests that require blood, fluid or tissue samples from the kiosk user. Used medical supplies are disposed of in safe disposal 400. Large screen TV 402 facilitates advertisements, announcements and the like, and stool/table 403 provide a comfortable user experience.

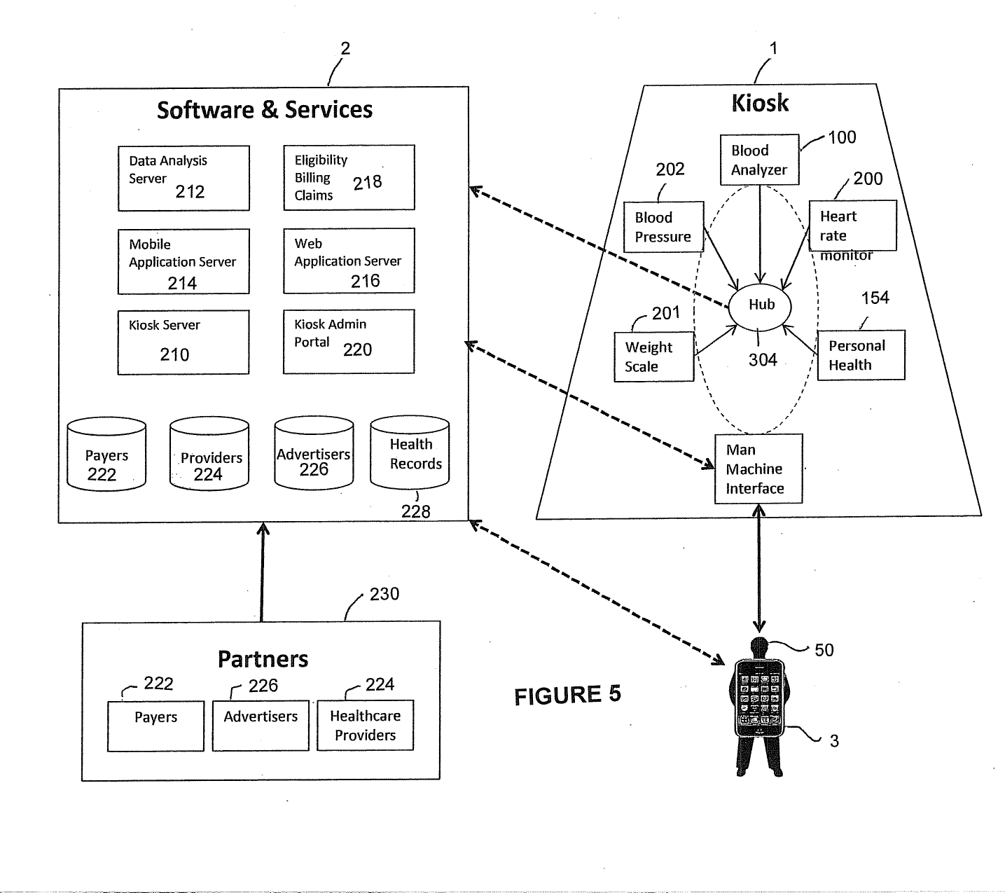

As shown in FIG. 5, blood/fluid/tissue analyzer 100, scale 201, blood pressure device 202 and heart rate monitor/oximeter 200 may be connected to hub 304 or to computer 305 using a wired connection (serial, USB or proprietary connector) or a wireless connection (WiFi, Bluetooth, ANT+ or any other wireless protocol). Hub 304 and/or computer 305 securely transmit the test measurement results from blood/fluid/tissue based test devices 10 and non-invasive test devices 20, along with personal health information 154 obtained from the user, to server 2 or the cloud for storage and/or further user consultation.

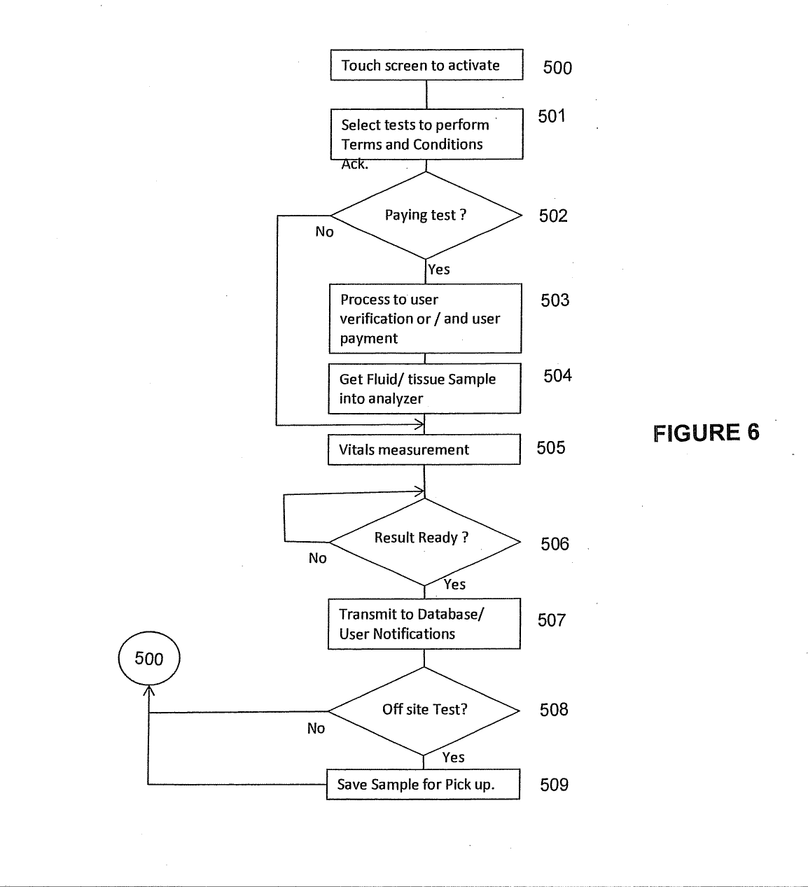

Server 2 may comprise multiple servers and components, as shown in FIG. 5. For example, server 2 may include kiosk server 210 that supports kiosk 1; data analysis server 212 that analyzes data received from kiosk 1 and/or user device 3; mobile application server 214 that supports a user interacting with server 2 via a mobile application residing on the user's smart phone or tablet; web application server 216 that supports a user interacting with server 2 via a web site; eligibility, billing and claims server 218 that supports payment and insurance eligibility aspects of the system, described below in more detail with reference to FIG. 6; and kiosk administration portal 220 for remote operator administration of kiosk 1.

Server 2 may also include databases of useful information such as payers 222, healthcare providers 224, advertisers 226 and health records 228. As shown in FIG. 5, server 2 may also receive data from outside partners 230 including, without limitation, payers 222, healthcare providers 224 and advertisers 226.

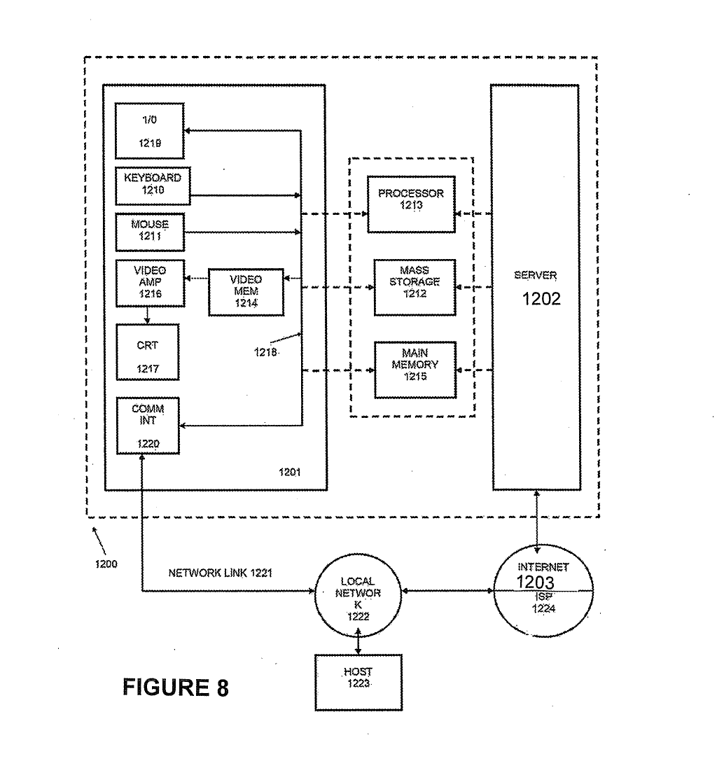

Kiosk software 307 guides the interaction between kiosk 1 and the kiosk user. Kiosk software 307 implements an algorithm that is depicted by the flowchart of FIG. 6. As described in more detail below with reference to FIG. 8, kiosk software 307 takes the form of computer-readable program code embodied in a non-transitory computer-readable medium that is executed by a processor of kiosk computer 305 and/or server 2. In step 500, the kiosk user touches touch screen 306 in order to begin interacting with kiosk 1. In step 501, touch screen 306 displays a list of tests available to be performed by blood/fluid/tissue based test devices 10 and non-invasive test devices 20. The costs of each test may also be displayed. The kiosk user makes a selection of tests, for example, by check marking desired tests. If a selection of a test is made that involves collection of blood/fluid/tissue, the kiosk user is guided to more specific blood/fluid/tissue test screen displays.

If a test selected by the kiosk user is a test that requires payment (step 502), then a process is initiated to verify the user and obtain payment (step 503). In particular, a registered kiosk user is asked to provide login information, whereas an unregistered user is asked to create an account. Insurance information and/or credit card information is obtained from the kiosk user for payment. If the kiosk user is insured, the kiosk user is invited to swipe an insurance card in card reader 303 or use other means to verify health insurance coverage. The kiosk user's insurance eligibility is checked and a co-pay amount (if any) is determined. If any co-payment or payment from an uninsured user is required, the kiosk user is invited to swipe a credit card in card reader 303.

After payment/insurance is processed, the test procedure begins (step 504). If blood/fluid/tissue collection is required, the kiosk user uses collection device 102 to provide a blood/fluid/tissue sample to collector 101 by a finger prick, and collector 101 is provided to analyzer 100 to begin blood/fluid/tissue analysis. Attendant 60, if present, can assist the kiosk user in these operations. While the blood/fluid/tissue is being analyzed, or if no blood/fluid/tissue analysis was required, the user is invited in step 505 to undergo tests for vitals measurements, such as by heart rate monitor/oximeter 200, scale 201 and/or blood pressure device 202. Attendant 60 if present can assist in these operations, and immediate results of these non-invasive tests may be displayed on touch screen 306.

In addition to blood/fluid/tissue sample collection and non-invasive tests, the kiosk user is invited to provide other personal health information. For example, the user may fill out a questionnaire on touch screen 306 including sex, age, health history, lifestyle, exercise habits and nutritional information. Software 307 combines assesses the results of the blood tests, vitals measurements and personal health information, and when the result is ready (step 506) provides feedback to the individual in the form of risk assessment, guidance and incentives for better health. For example, the kiosk user may be provided with an estimate of the risk for coronary heart disease. Software 307 may generate lifestyle change recommendations such as exercise and diet plans, and may recommend products and services useful for implementing the lifestyle change recommendations. Software 307 may also generate coupons or vouchers for recommended products and services.

In step 507, the test results are aggregated and transferred to server 2 or the cloud, along with an electronic notification to the user. A hard copy of all results may also be printed at this stage. The measurement data and test results may also be transferred or pushed to In step 508, if the user has requested a test that cannot be performed at kiosk 1, such as genomic/genotyping testing, collector 101 that includes the collected sample is placed in a safe deposit for later pickup and shipment to a professional laboratory (step 509) for more complex analysis. Otherwise, the sample is moved to safe disposal 400 and kiosk 1 is ready for the next kiosk user.

The test results along with guidance for lifestyle changes may also be provided by server 2 and/or kiosk 1 to user device 3 via Internet connection 23. A mobile application executing on user device 3 may display the test data and test result history, and may provide other options such as sharing information with a medical network or a physician; storing data in a personal health record; and making an appointment with a physician, who will be able to access the recorded health record of the consumer. The application may also display advertisements of products and services that are of relevance to the user's health situation, as well as trends and progress made by the user.

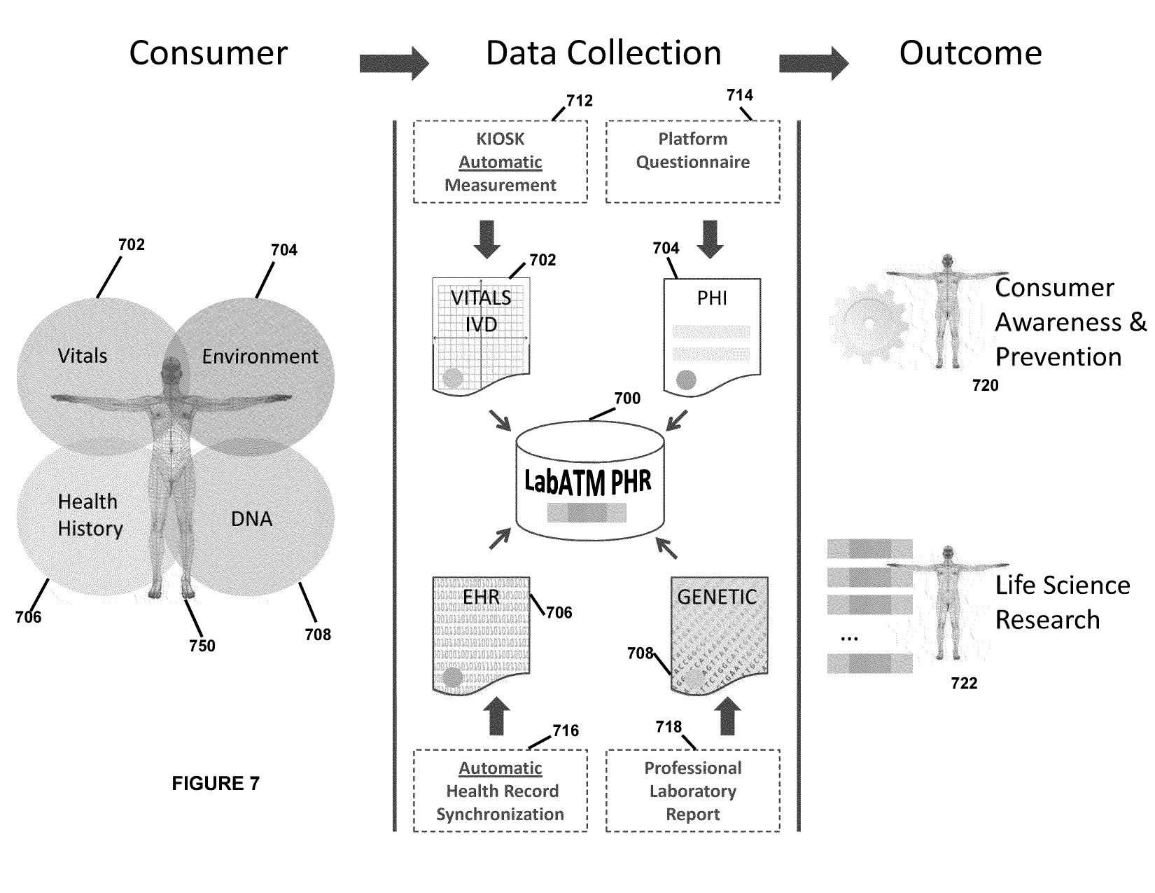

FIG. 7 is a conceptual diagram showing the data collection and flow that occurs during execution of the algorithm of FIG. 6. The kiosk user provides vitals and IVD data from blood/fluid/tissue samples 702. Data 702 is obtained by automatic measurement and testing 712 at kiosk 1. In addition, the user provides personal health information (PHI) and environmental/lifestyle information 704. Information 704 may be obtained, for example, by user completion of questionnaire 714 on touch screen 306 of kiosk 1. The health history/electronic health records (EHR) 706 of the kiosk user may be obtained by automatic health record synchronization with external sources such as electronic medical record systems 716. Genetic (DNA) information 708 may be obtained from a professional laboratory report 718 of an outside laboratory.

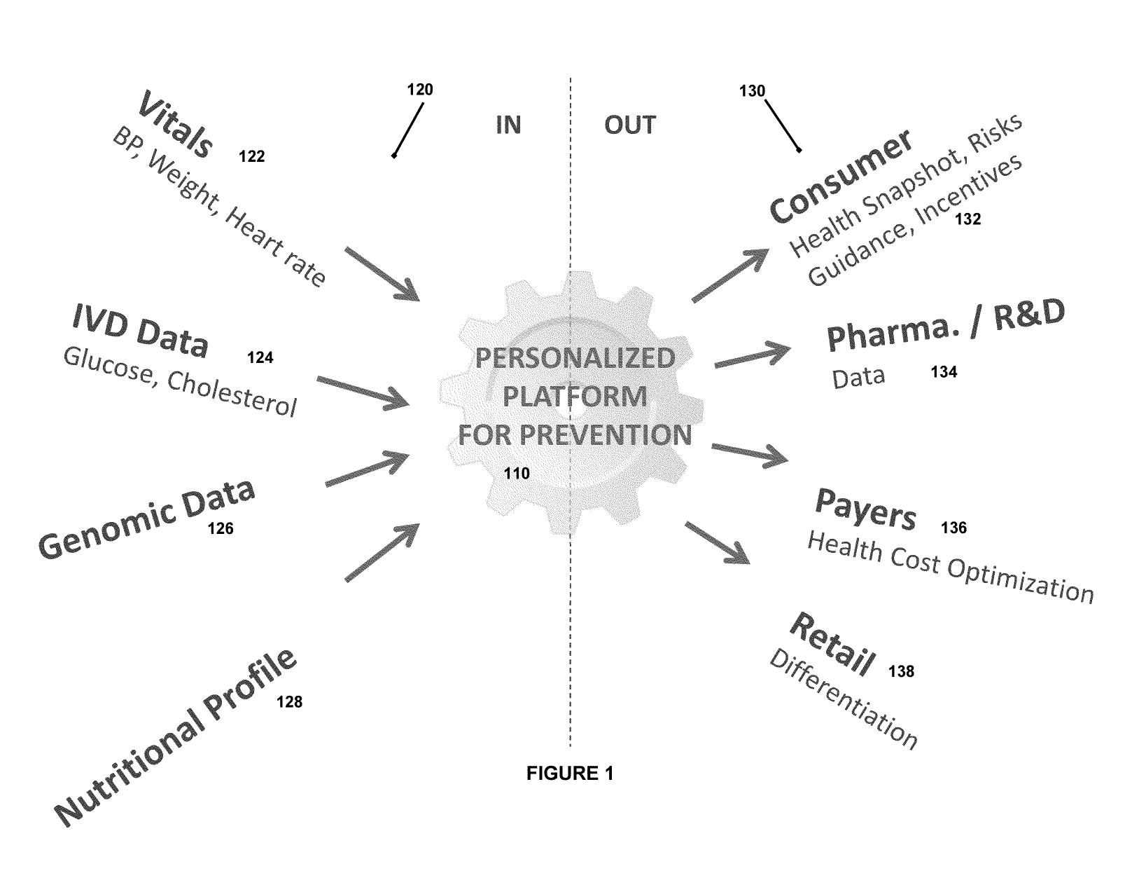

These sources of information 702, 704, 706, 708 are analyzed by platform 700 of the present invention (i.e. kiosk 1 and/or server 2), resulting in generation of output data 720 relevant to consumer awareness and/or prevention as well as output data 722 relevant to life science, medical and pharmaceutical research. Output data 720 may include, for example, test results, a health snapshot risk assessments, guidance such as exercise and diet plans, recommended products and services, and user incentives such as coupons or vouchers for recommended products and services. In addition to output data 720 and 722, and with reference to FIG. 1, output data of interest to payers and retailers may also be generated.

Various components of the platform of the present invention, including the electronics control devices and software 30 of kiosk 1, server 2 and user device 3, may be implemented in a general purpose computing environment 1200 such as that illustrated in FIG. 8. Kiosk software 307, for example, may be implemented as computer-readable program or application code that is executed by processor 1213 in computing environment 1200. Computer-readable code may be embodied in a computer program product or non-transitory computer-readable medium configured to store or transport computer-readable code, or in which computer-readable code may be embedded. Examples of computer program products and non-transitory computer-readable mediums include, without limitation, computer memories and hard drives (RAM, ROM, etc.), semiconductor memory devices, hard and floppy disks, magnetic tapes, optical disks, servers on a network, and so on.

Computer 1201 of computing environment 1200 may be a laptop, desktop, tablet, smartphone, or other processing device. As such, computer 1201 represents one possible configuration of computer 305 of kiosk 1, as well as a possible configuration of user device 3. Computer 1201 may include, without limitation, keyboard 1210, mouse 1211, mass storage 1212, processor or central processing unit (CPU) 1213, video memory 1214, main memory 1215, video amplifier 1216, display 1217 and communication interface 1220, all coupled to bi-directional system bus 1218.

Keyboard 1210 and mouse 1211 introduce user input to the system and communicate that input to processor 1213 via bus 1218. Other suitable input devices (such as touch screen 306, for example) may be used in addition to, or in place of, keyboard 1210 and mouse 1211. Input/output (I/O) unit 1219 is also coupled to bus 1218 and generally represents any other element or apparatus that may provide input to and receive output from computer 1201 such as, for example, printer 301, camera 302 and card reader 303 of kiosk 1.

Processor 1213 may be any suitable microprocessor including a cloud computing solution. Processor 1213 may reside on computer 1201, server 1202 or, as indicated by the dashed lines in FIG. 8, may have its computational power distributed between computer 1201 and server 1202. Computer 1201 and server 1202 may each have its own processor. As with processor 1213, main memory 1215 and mass storage 1212 may reside on one or both of server 1202 and computer 1201, or may be distributed between the two. Examples of systems where processor 1213, main memory 1215 and mass storage 1212 are distributed between computer 1201 and server 1202 include thin-client computing architectures and other personal digital assistants, Internet ready cellular phones and other Internet computing devices, and in platform independent computing environments.

Main memory 1215 and mass storage 1212 may include both fixed and removable media, such as magnetic, optical or magnetic optical storage systems or any other available mass storage technology. Main memory 1215 and video memory 1214 may comprise, for example, random access memory (RAM) or dynamic random access memory (DRAM). Video amplifier 1216 converts data stored in video memory 1214 to a signal suitable for display on display 1217.

Communication interface 1220 provides two-way data communication via network link 1221 to local network 1222. For example, if communication interface 1220 is an integrated services digital network (ISDN) card or a modem, communication interface 1220 provides a data communication connection to the corresponding type of telephone line, which comprises part of network link 1221. If communication interface 1220 is a local area network (LAN) card, communication interface 1220 provides a data communication connection via network link 1221 to a compatible LAN. Communication interface 1220 may also provide a wireless communication link. In any such implementation, communication interface 1220 sends and receives electrical, electromagnetic or optical signals that carry digital data streams representing various types of information. Network link 1221 provides data communication through one or more networks to other data devices. For example, network link 1221 may provide a connection via local network 1222 to local server computer (host) 1223, or may provide a connection via Internet service provider (ISP) 1224 and Internet 1203 to server 1202.

Computing environment 1200 is merely one exemplary framework of a computing environment in which the present invention may be implemented. The present invention may be implemented in any suitable computing environment including personal computers, tablet computing devices, smart phones and the like.

1. A self-serve kiosk comprising:
a collection device for obtaining a blood, fluid or tissue sample from a user;
a collector for collecting the blood, fluid or tissue sample;
an analyzer for analyzing the blood, fluid or tissue sample; and
a user interface that provides test results of analyzing the blood, fluid or tissue sample.

2. The self-serve kiosk of claim 1, wherein the user interface obtains input of personal health information from the user, and further comprising:
a non-invasive test device for obtaining vitals measurements from the user; and
a processor that analyzes the blood, fluid or tissue sample, vitals measurements, and personal health information, and outputs test results indicative of the health of the user.

3. The self-serve kiosk of claim 1, wherein the collection device comprises a finger stick device for obtaining blood from the user.

4. The self-serve kiosk of claim 1, further comprising a safe waste for storing contaminated collection devices.

5. The self-serve kiosk of claim 1, further comprising a safe for storing the collected blood, fluid or tissue sample.

6. The self-serve kiosk of claim 1, wherein the non-invasive test device comprises at least one of a weight scale, a blood pressure device and a heart rate monitor/oximeter.

7. The self-serve kiosk of claim 1, wherein the user interface is a touch screen.

8. The self-serve kiosk of claim 1, further comprising a card reader for obtaining insurance and payment information from the user.

9. The self-serve kiosk of claim 1, further comprising a dispenser module for dispensing cash and/or for dispensing coupons or vouchers for products or services of relevance to the health of the user.

10. The self-serve kiosk of claim 1, further comprising an Internet connection hub for transmission of the test results to a server or a user device.

11. A system comprising the self-serve kiosk, the server and the user device of claim 10.

12. The self-serve kiosk of claim 1, wherein the test results include at least one of measurements, a health snapshot, a risk assessment, and guidance for improving health.

13. A non-transitory computer-readable medium storing program code that causes a processor to execute operations, at a self-serve kiosk, comprising:
displaying tests available to be performed at the kiosk;
obtaining from a selection of a test from a user;
obtaining insurance information from the user;
determining insurance eligibility of the user for the selected test;
determining whether additional payment is required;
obtaining any required additional payment from the user;
performing the selected test; and
providing results of the test to the user.

14. The non-transitory computer-readable medium of claim 13, wherein the available tests include a blood, fluid or tissue analysis test that is performed automatically by collecting blood, fluid or tissue from the user.

15. The non-transitory computer-readable medium of claim 14, wherein the program code causes the processor to execute a further operation comprising:
analyzing the collected blood, fluid or tissue and generating in vitro diagnostic data.

16. The non-transitory computer-readable medium of claim 14, wherein the program code causes the processor to execute a further operation comprising:
storing the collected blood, fluid or tissue for shipment to a professional laboratory for complex analysis including genotyping.

17. The non-transitory computer-readable medium of claim 14, wherein the available tests further comprise a non-invasive test for vitals measurements.

18. The non-transitory computer-readable medium of claim 17, wherein the non-invasive test measures at least one of weight, blood pressure and heart rate.

19. The non-transitory computer-readable medium of claim 13, wherein the program code causes the processor to execute a further operation comprising:
transmitting the results of the test to a server for further analysis and storage.

20. The non-transitory computer-readable medium of claim 13, wherein the program code causes the processor to execute a further operation comprising:
transmitting the results of the test to a mobile application on a user device.

21. The non-transitory computer-readable medium of claim 13, wherein the program code causes the processor to execute further operations comprising at least one of:
providing a risk assessment to the user based on the test results;
providing a guidance plan to the user based on the test results; and
providing an incentive to the user to follow the guidance plan.

22. The non-transitory computer-readable medium of claim 21, wherein the incentive comprises a coupon of relevance to a health condition of the user.

23. The non-transitory computer-readable medium of claim 21, wherein the incentive comprises cash.
